(19) United States
(12) Patent Application Publication
(10) Pub. No.: US 2012/0184886 A1
(43) Pub. Date: Jul. 19, 2012

(54) RELIEF MEMBER

(75) Inventors: David Huber, Austinmer (AU); Craig Andrews, Kirribilli (AU)

(73) Assignee: GUARDAHEEL IP PTY LTD, Kirribilli (AU)

(21) Appl. No.: 13/375,862

(22) PCT Filed: Jun. 4, 2010
(86) PCT No.: PCT/AU2010/000696

§ 371 Date: Apr. 2, 2012

(30) Foreign Application Priority Data
Jun. 5, 2009 (AU) 2009902622

(51) Int. Cl. A61F 5/01; B23P 17/04

(52) U.S. Cl. 602/5; 29/428

(57) ABSTRACT

A relief member is described. The relief member is suitable for engaging a support member adapted to support a subject's body part and includes a relief member body which includes a body thickness and comprises a relief member body plane disposed along a selected body axis; and an engagement means integral with the relief member body and adapted to engage with a support member, the engagement means being movable between a first configuration and an engagement configuration, the engagement means in the first configuration being disposed within extremity boundaries of the body thickness or is disposed substantially within the same plane as the relief member body plane, and the engagement means when in the engagement configuration extending outside the extremity boundaries of the body thickness or outside the relief member body plane to releasably engage the relief member to the support member.

### TECHNICAL FIELD

The present invention is broadly directed to means of engaging an attachable or removable member to an object. Preferably, the member is a relief member adapted to provide pressure relief to one object resting on or adjacent another. More specifically, the present invention is directed to such relief members further adapted to engage at least one such object. More specifically still, the present invention is directed to such relief members further adapted to provide pressure relief to a subject's body part resting on or adjacent an object, such as a support member or orthosis.

### BACKGROUND

Use of relief members, such as cushioning means, pillows, foam pieces, gel blocks and the like, to provide pressure relief for one object resting on or adjacent another is known. In circumstances involving two or more inanimate objects resting on or adjacent one another, providing pressure relief to the objects may relatively straightforwardly involve interposing a relief member between the objects and relying on the respective weights of the objects, the inertia between them or the opposing forces they impose on one another (in accordance with Newton's laws of motion), to retain the relief member in a suitable position to provide the requisite pressure relief.

The problem of retaining the relief member in such suitable position is not straightforward when one or more of the objects is not inanimate. One example of such circumstances is where one of the objects is a subject's body part and the other is a support member, for example, an orthosis. The problem becomes more complex in these circumstances when it is desirable to refrain from securing the orthosis to the body part, thereby eliminating any potential mechanism to retain a relief member suitably interposed between the body part and the orthosis at or in the vicinity of a desired pressure relieving site, by effectively ‘sandwiching’ the relief member between the body part and the orthosis.

Even in some circumstances where the orthosis is secured to the body part, by a strap for example, the problem of retaining a relief member at a desired pressure relieving site can be difficult. Consider the scenario where the orthosis is a calliper adapted to maintain a subject's weak or paralysed leg in extension to enable the subject to walk. Motion of the subject's leg consistent with gait typically tends to result in relative movement between the leg and calliper even in spite of the strap. A degree of movement between the leg and the calliper may, in any event, be a functional condition precedent to facilitate walking. Ultimately, this relative movement creates periods where there is no or minimal pressure between some parts of the leg and some parts of the calliper rendering it not possible to rely on the respective weights of the leg and calliper, the inertia between them or the opposing forces they impose on one another, to retain the relief member at or in the vicinity of a desired pressure relieving site.

In order to address these difficulties, relief members themselves are typically secured to the orthosis and/or to the subject's body part, at locations where providing pressure relief is or may be necessary. In this way, the fact that one of the objects for which pressure relief may be required is not inanimate, does not prevent or inhibit pressure relief from being provided at desired pressure relieving sites.

Mechanisms for securing the relief member to a subject's body part are limited, particularly where the subject's body part is bare when it comes into contact with the orthosis. In some such cases, a mild, non-irritating adhesive can be used to secure the relief member to the body part, but where multiple relief members must be used, it is cumbersome and inconvenient to apply them all. Although securing a relief member to a stocking or other such barrier material which can be worn by the body part may be possible, this also requires an adhesive or another mechanism, such as sewing the relief member to the barrier material. If there is relative movement between the barrier material and the body part or orthosis, the relief member may not be suitably positioned to provide the desired pressure relief. Furthermore, additionally requiring the wearing of such a barrier material creates potentially unnecessary inconveniences.

There is a wide variety of known mechanisms to give effect to securing a relief member to an orthosis. These include adhesives, such as glue, double-sided tape, rivets, nails, screws and such other more permanent securing mechanisms.

One of the problems or disadvantages with more permanent securing mechanisms is that they render it difficult to remove and replace relief members as they become soiled or they otherwise deteriorate. It is also unhygienic, and in many cases, impermissible to bring relief members used by one subject into contact with another subject, particularly in the clinical setting. Accordingly, by using more permanent securing mechanisms to secure a relief member to an orthosis effectively limits the use of each orthosis to one patient only, and invariably increases the turnover of orthoses and generation of waste.

Another problem or disadvantage with known mechanisms for securing relief members to body parts or orthoses relates to the requirement of multiple types of materials to be employed. In each mechanism discussed above, there is at least two types of materials required before the relief member can be secured to the orthosis—the type of material/s from which the relief member is formed and the type of material used to secure the relief member to the orthosis. The use of multiple material types increases cost of manufacture, both in terms of parts and labour, and complicates ultimate assemblage for the end user.

The present inventors have developed an improved means of attaching a relief member for an object such as a support.

### SUMMARY OF THE INVENTION

According to a first aspect, the present invention provides a relief member for engaging a support member adapted to support a subject's body part, comprising:
a relief member body which includes a body thickness and comprises a relief member body plane disposed along a selected body axis; and
an engagement means integral with the relief member body and adapted to engage with a support member,
the engagement means being movable between a first configuration and an engagement configuration, the engagement means in the first configuration being disposed within extremity boundaries of the body thickness or is disposed substantially within the same plane as the relief member body plane, and the engagement means when in the engagement configuration extending outside the extremity boundaries of the body thickness or outside the relief member body plane to releasably engage the relief member to the support member.

Preferably the engagement means includes an integral tongue or tab.

Preferably, in use the engagement means when in the engagement configuration extends or moves into a recess on the support member to releasably engage the relief member to the support member.

In some such preferred embodiments, the relief member body has a first relief surface and a second relief surface substantially opposite the first relief surface, and the body thickness is between the first and second relief surfaces. The body thickness may be consistent or may vary. Preferably, in use, the first relief surface is adapted to rest on or adjacent the subject body part and the second relief surface is adapted to rest on or adjacent the support member.

The engagement means of some such preferred embodiments has a first engagement surface, a second engagement surface substantially opposite the first engagement surface and an engagement thickness between the first and second engagement surfaces. The engagement thickness may be consistent or may vary.

In some preferred embodiments, when the engagement means is in the first configuration, the first engagement surface is substantially flush with the first relief surface, or locates within the extremity boundaries of the body thickness, and the second engagement surface is substantially flush with the second relief surface, or locates within the extremity boundaries of the body thickness. When the engagement means of such embodiments is changed to the engagement configuration, either or both of the engagement surfaces and at least part of the engagement thickness, are caused to partly protrude from, or otherwise exceed, the extremity boundaries of the relief member body thickness, or to move into a different plane to the relief member body plane.

Typically, when in the engagement configuration, at least part of the engagement means protrudes from, or otherwise exceeds, at least one of the first or second relief surfaces. In a particularly preferred embodiment, at least part of the engagement means protrudes from, or otherwise exceeds, the second relief surface. In some such embodiments when the engagement means is in the engagement configuration, the first relief surface is substantially planar with a depression or opening where the first engagement surface otherwise locates when the engagement means is in the first configuration. This physical arrangement for the engagement means in the engagement configuration is preferred for embodiments where the first relief surface is adapted to rest on or adjacent the subject body part.

The preferred plan view shape for the relief member body may be determined by a number of factors including physical features of the anatomical region over which pressure relief is to be provided to the subject body part, features of shape and/or configuration of the support member, of part of the support member which, in use, may be prone to providing undue pressure to a particular body part, or of a relief region in the support member specifically formed to accommodate a relief member. There may also be other additional factors for determining the shape of the relief member body which, depending on the particular circumstances, may be known to the skilled addressee. Moreover, the relief member body of preferred embodiments may have any suitable shape.

In some preferred embodiments, for example, the relief member body has a shape and/or configuration which substantially corresponds to a shape and/or configuration of at least a portion of a relief region formed in the support member to accommodate a relief member.

Preferably, at least a portion of the relief member body is configured so as to contour at least a portion of the relief region when the relief member is located on or in the vicinity of the relief region. In one preferred embodiment, for example, the relief member is key shaped and is adapted to contour at least a portion of the relief region which is correspondingly shaped. In another preferred embodiment, the relief member body is a composite shape made up of two or more shapes, such as, for example, a rectangle adjacent a semi-circle or a rectangle adjacent a part-circle, greater or lesser in area than a semi-circle with the same diameter. In such embodiments, the semi-circle or part-circle is adjacent to a short side of the rectangle and in other embodiments, the semi-circle or part-circle is adjacent the longer side of the rectangle.

The diameter of the semi-circle or the part-circle may be equal to, shorter than or longer than the length of the side of the rectangle to which it is adjacent. In a particularly preferred embodiment, the shape of the relief member is comprised of a part-circle, lesser in area than a semi-circle with the same diameter, adjacent a long side of a rectangle, wherein the diameter of the circle is shorter than the long side of the rectangle and the meeting point of the circumference of the part-circle with the long side of the rectangle is smooth and curved.

The relief member body of some preferred embodiments is preferably formed from one or more materials selected from the group consisting of low density foam, high density foam, open cell or closed cell foams, rubber, solid or semi-solid gels and a combination of two or more thereof.

The relief member body of other preferred embodiments can be formed of any suitable material, whether natural or synthetic. For example, in some embodiments, the relief member body may be formed of one or more pliable enclosures, with each pliable enclosure adapted to receive, hold or expel relief member density content.

In some such embodiments, the pliable enclosure is preferably formed of one or more materials selected from the group consisting of polyurethane, resins, elastomers, polymers, copolymers, pliable plastics, leather, rubber and a combination of two or more thereof. The pliable enclosure of other preferred embodiments can be formed of any suitable material, whether natural or synthetic.

The relief member density content of some preferred embodiments may be selected from the group consisting of air, gas, gel, water, liquid, beads, foam particles or pieces, feathers, pulses or seeds of any kind, and a combination of two or more thereof. The relief member density content of other preferred embodiments can be formed of any suitable material, whether natural or synthetic.

The relief member body may also be formed of a combination of the materials identified above and of one or more pliable enclosures. Some embodiments enable variation in the density of the relief member body.

In particularly preferred embodiments, the relief member body is formed of an open cell foam. As the engagement means is integral with the relief member body, the engagement means is preferably formed of the same material.

In a particularly preferred embodiment, the engagement means is formed as tongue-like structure adapted to be changeable between the first and the engagement configurations. The engagement means may be formed as an extension from the perimeter of the relief member body or from one or both of the first or second relief surfaces. In particularly preferred embodiments, the engagement means is preferably partly formed by virtue of a part- or full-thickness division through the relief member body. Preferably, the division is full-thickness spanning the first to the second relief surface.

The formation of the division is determined depending on the material/s from which the relief member body is formed. For example, where the relief member body is formed of a pliable enclosure, the division defining the engagement means may be formed as a saccular-type division which may or may not retain communication of flow of relief member density content between the engagement means and the relief member body. In particularly preferred embodiments, wherein the relief member body is formed of an open cell foam, the division is formed by a cut through the foam. The invention envisages other means for forming the division to create the engagement means.

In preferred embodiments, the shape of the division determines the plan view shape of the engagement means or of the first and/or second engagement surfaces. As the spatial arrangement of the engagement means in the engagement configuration is adapted so as to releasably engage the relief member to the support member, the shape of the division may contribute to the quality and quantity of that engagement.

In some preferred embodiments, the shape, or a part of the shape, of the division is circular, ovular, rectangular, triangular, linear, elongated, elongated with a portion which is part circular, part ovular, part rectangular, part triangular, part linear, a composite shape comprising two or more shapes, or combinations of these. The invention envisages a wide range of shapes for the division.

In preferred embodiments, releasable engagement of the relief member to the support member is preferably achieved when the engagement means is changed to the engagement configuration and advanced into an orifice, lacuna, or channel in the support member. Preferably, the shape of the division defining at least part of the engagement means may be partially determined by reference to the shape of the orifice, lacuna, or channel into which the engagement means will be advanced when in the engagement configuration.

The spatial arrangement of the engagement means in the engagement configuration releasably engages the relief member to the support member, in some preferred embodiments by applying an outward pressure to an interior of the orifice, lacuna, or channel formed in the support member. In some preferred embodiments, wherein the relief member body and the engagement means are formed from foam, for example an open cell foam, the releasable engagement is preferably achieved as the engagement means is caused to advance into the orifice, lacuna, or channel of the support member (taking up the engagement configuration) and the expansile properties of the foam generate a spatial arrangement for the engagement means which applies the outward pressure to the orifice, lacuna, or channel interior. In this way, the relief member is releasably engaged to the support member as the outward pressure applied to the orifice, lacuna, or channel interior by the spatial arrangement of the engagement means in the engagement configuration generates inertial forces capable of releasably retaining the engagement means in the orifice, lacuna, or channel.

In other preferred embodiments, the spatial arrangement of the engagement means in the engagement configuration releasably engages the relief member to the support member by other mechanisms. For example, in some preferred embodiments, the shape of the division defining at least part of the engagement means is adapted to maximise volume of the engagement means in the engagement configuration. The greater the volume of the engagement means in the engagement configuration, the greater the amount of engagement means material that can be advanced (or forced) into the orifice, lacuna, or channel. In this way, a ‘force-fit scenario’ is created in which the engagement means is effectively releasably wedged into the orifice, lacuna, or channel and the relief member is thereby releasably engaged to the support member. In some such embodiments, the division defining at least a part of the engagement means is a partial thickness division. In other such embodiments, the division defining at least a part of the engagement means is a full thickness division.

In some preferred embodiments, the relief member comprises at least two engagement means integral with the relief member body, adapted to engage the support member.

The engagement means can be formed at any suitable location in or on the relief member body. In one preferred embodiment, an engagement means is formed substantially centrally on the plan view of the relief member body. In other embodiments, the engagement means is/are formed toward the perimeter of the plan view of the relief member body. The invention envisages a wide range of suitable locations where the engagement means is/are formed.

Preferably, the engagement means is/are generally biased toward the first configuration.

In some preferred embodiments, the relief member is disposable.

In some particularly preferred embodiments, the support member is an orthosis.

According to a second aspect, the present invention provides a relief member comprising:
a relief member body; and
an engagement means integral with the relief member body,
wherein the engagement means is configurable to an engagement configuration adapted so as to releasably engage the relief member to an object.

Preferably, the engagement means of the relief member of the second aspect is changeable from a first configuration to the engagement configuration.

In preferred embodiments, the engagement configuration of the engagement means of the relief member of the second aspect has a spatial arrangement adapted to provide an outward pressure against an interior of an orifice, lacuna, or channel in the object.

Advantageously, some preferred embodiments of the relief member provide a releasable engagement which allows for some movement between the relief member and the support member. This inhibits “shear”, which is one of the causes of pressure ulcers. This movement is facilitated by the integral engagement tongues or tabs being only a portion of the surface of the main body of the relief member body.

According to a third aspect, the present invention provides a method of forming a relief member according to the first or second aspects of the invention, the method comprising:
forming a relief member body; and
forming a division in the relief member body adapted to form at least part of an engagement means.

In a preferred embodiment, the division is a part- or full- thickness division through the relief member body. Preferably, the division is full-thickness spanning from one surface of the relief member body to an opposite surface of the relief member body. In some preferred embodiments, the division is created by a cut.

According to a fourth aspect, the present invention provides a relief member formed according to the method of the third aspect of the invention.

According to a fifth aspect, the present invention provides a method of releasably engaging a relief member to an object, the method comprising:
providing a relief member according to the first, second or fourth aspects of the present invention;
providing an orifice, lacuna, or channel in the object; and
advancing the engagement means into the orifice, lacuna, or channel so as to adopt the engagement configuration thereby releasably engaging the relief member to the object.

According to a sixth aspect, the present invention provides a method of releasably engaging a relief member to an object, the method comprising:
providing a relief member according to the first, second or fourth aspects of the present invention;
providing an orifice, lacuna, or channel in the object;
causing the engagement means to adopt the engagement configuration; and
advancing the engagement means in the engagement configuration into the orifice, lacuna, or channel thereby releasably engaging the relief member to the object.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention.

In order that the present invention may be more clearly understood, preferred embodiments will be described with reference to the following drawings and examples.

### BRIEF DESCRIPTION OF THE DRAWINGS

FIG. 1A is a plan view of a preferred embodiment of a relief member according to the present invention. FIG. 1B is a side elevation of the embodiment of the relief member depicted in FIG. 1A, illustrating in dotted lines where a division defining an engagement member may be located. FIG. 1C is a cross-sectional elevation of the embodiment of the relief member depicted in FIG. 1A illustrating the engagement means in the engagement configuration.

FIG. 2 is a perspective disassembled view illustrating a relief member according to a preferred embodiment of the invention positioned for engagement with an orthosis.

FIG. 3 is a perspective view of the relief member and orthosis depicted in FIG. 2 illustrating the relief member engaged with the orthosis.

FIGS. 4A and 4B are cross sectional views respectively illustrating the relief member and orthosis as depicted in FIGS. 2 and 3. FIG. 4A illustrates the relief member with an engagement means in the first configuration and FIG. 4B illustrates the relief member with the engagement means in the engagement configuration.

FIGS. 5A and 5B are transverse sectional views respectively corresponding to the illustrations of the relief member and orthosis as depicted in FIG. 4A and 4B.

FIGS. 6A and 6B are perspective views respectively illustrating: a relief member according to yet another embodiment of the present invention positioned for engagement with an orthosis; and the same relief member engaged with the orthosis in accordance with the present invention.

FIG. 7A is a plan view of another preferred embodiment of a relief member according to the present invention, illustrating in dotted lines where a division defining an engagement member may be located. FIG. 7B is a side elevation of the embodiment of the relief member depicted in FIG. 7A, illustrating in dotted lines where the division defining the engagement member is located. FIG. 7C is a cross-sectional elevation of the embodiment of the relief member depicted in FIG. 7A illustrating the engagement means in the engagement configuration.

FIGS. 8A and 8B are perspective views respectively illustrating: the relief member depicted in FIGS. 7A to 7C positioned for engagement with an orthosis; and the same relief member engaged with the orthosis in accordance with the present invention.

FIGS. 9A and 9B are perspective views respectively illustrating: a relief member according to yet another embodiment of the present invention positioned for engagement with an orthosis; and the same relief member engaged with the orthosis in accordance with the present invention.

FIGS. 10A and 10B are perspective views respectively illustrating: a relief member according to yet another embodiment of the present invention positioned for engagement with an orthosis; and the same relief member engaged with the orthosis in accordance with the present invention.

FIG. 11 is a schematic line drawing illustrating a subject's leg resting on an orthosis with a relief member according to one embodiment of the present invention engaged with the orthosis.

### DETAILED DESCRIPTION OF PREFERRED EMBODIMENTS

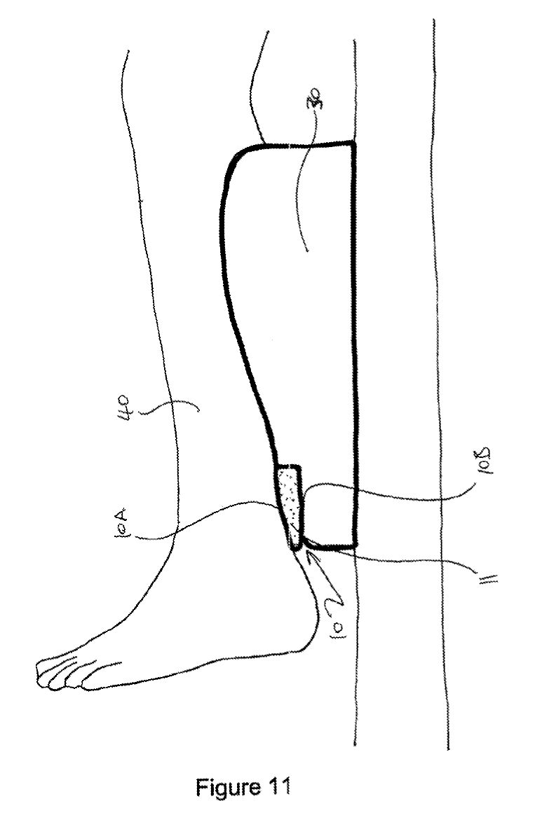

A relief member 10 according to the present invention can be used to provide pressure relief for one object resting on or adjacent another. In particular preferred embodiments, the relief member 10 is used to provide pressure relief for a subject body part, such as a leg 40, resting on or adjacent a support member 30, such an orthosis. FIG. 11 provides a simplified schematic line drawing illustrating a subject's leg 40 resting on an orthosis 30 with a relief member 10 according to one embodiment of the present invention engaged with the orthosis 30.

The figures are principally directed to embodiments of a relief member 10 adapted for use with an orthosis 30. For consistency, the same general form of orthosis 30 is used in each of the figures. However, a person skilled in the art would readily appreciate that a relief member 10 according to various embodiments of the present invention could be used with a wide range of orthoses, bearing a number of different features.

As illustrated in the Figures generally, relief member 10 comprises a relief member body 11 and an engagement means 12 integral with the relief member body 11 adapted to engage with the orthosis 30. The engagement means 12 is changeable between a first configuration and an engagement configuration, wherein the spatial arrangement of the engagement means 12 in the engagement configuration is adapted so as to releasably engage the relief member 10 to the orthosis 30.

Relief member body 10 has a relief member body plane 10D. Preferably, when in the first configuration, the engagement means 12 is in substantially the same plane as the relief member body plane 10D, or substantially within extremity boundaries of the body thickness 10C of the relief member body 11. Preferably, when in the engagement configuration, the engagement means 12 is in a different plane to the relief member body plane 10D, or at least partly protrudes from, or otherwise exceeds, extremity boundaries of the relief member 10 body thickness 10C.

Relief member body 11 has a first relief surface 10A and a second relief surface 10B (substantially opposite the first relief surface 10A), and the body thickness 10C spans a distance between the first and the second relief surfaces 10A and 10B. The body thickness 10C maybe consistent or may vary. Relief members 10 according the varying embodiments illustrated in the Figures generally show the body thickness 10C as being consistent. Preferably, in use, the first relief surface 10A is adapted to rest on or adjacent the subject body part and the second relief surface 10B is adapted to rest on or adjacent the orthosis 30.

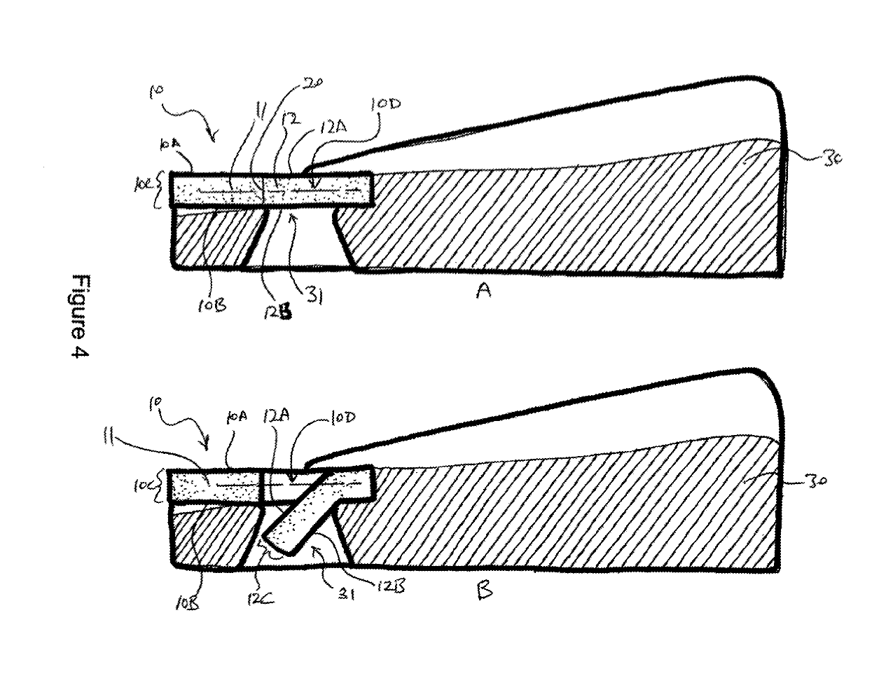

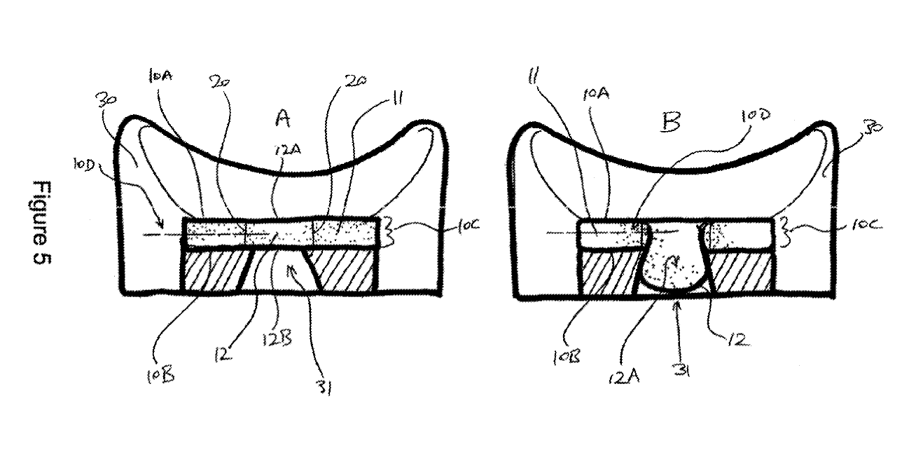

The engagement means 12 has a first engagement surface 12A, a second engagement surface 12B (substantially opposite the first engagement surface 12A) and an engagement thickness 12C which spans a distance between the first and second engagement surfaces 12A and 12B. The engagement thickness 12C maybe consistent or may vary. As is well illustrated in cross sectional views in FIGS. 4A and 5A depicting a particular embodiment of relief member 10 according to the present invention, when engagement means 12 is in the first configuration, the first engagement surface 12A is substantially flush with the first relief surface 10A, and the second engagement surface 12B is substantially flush with the second relief surface 10B. When engagement means 12 of such embodiments is in the engagement configuration, either or both of engagement surfaces 12A and 12B and at least part of engagement thickness 12C, are caused to partly protrude from, or otherwise exceed, the extremity boundaries of the relief member 10 body thickness 10C. This is well illustrated in cross sectional views in FIGS. 4B and 5B.

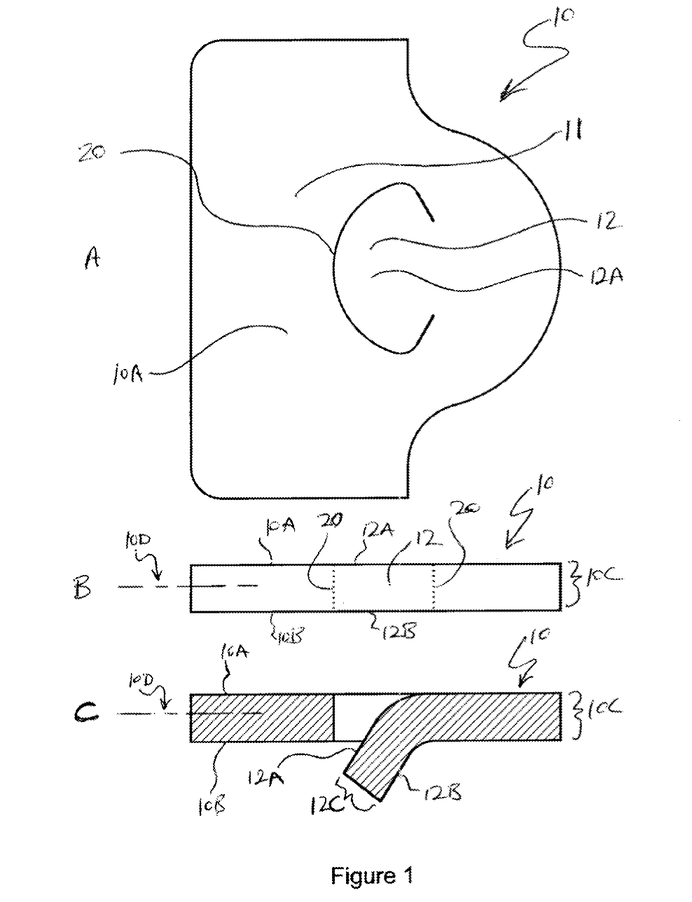

One preferred plan view shape of the relief member body 11 is well illustrated in FIG. 1A, and also in other diagrams. The preferred plan view shape may be determined by a number of factors including the physical features of the anatomical region over which pressure relief is to be provided to the subject body part, features of shape and/or configuration of the orthosis 30 or of parts of the orthosis 30 which, in use, may be prone to providing an undue pressure to a particular body part, or of a relief region 32 in the orthosis 30, specifically formed to accommodate a relief member 10. Moreover, the relief member body 11 of preferred embodiments may have any suitable shape, including plan view shape.

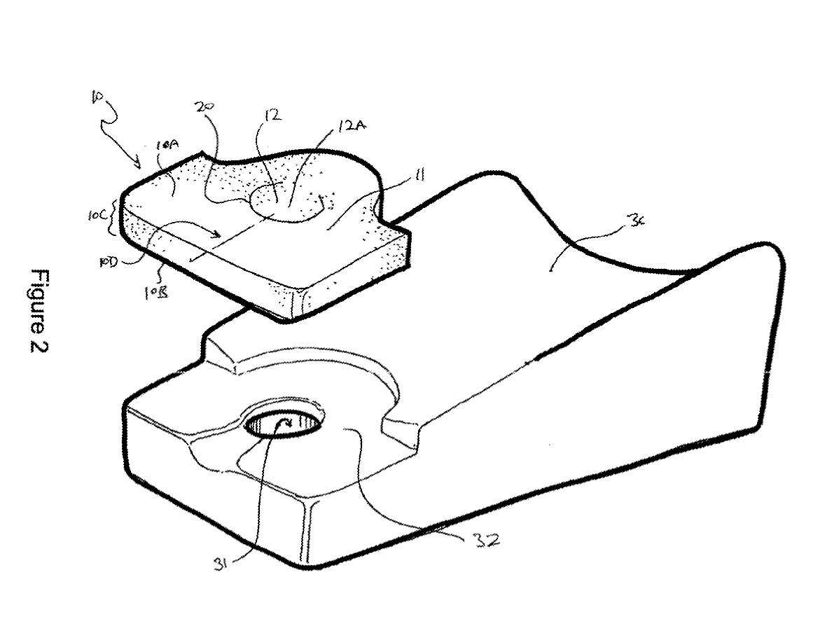

As illustrated in FIGS. 2, 3, 6A and 6b, 8A and 8B, and 9A and 9B, the relief member body 11 has a shape and/or configuration which substantially corresponds to a shape and/or configuration of at least a portion of a relief region 32 formed in the orthosis 30 to accommodate a relief member 10.

The plan view depicted in FIG. 1A well illustrates that, in some preferred embodiments, the relief member body 11 is a composite shape made up of two or more shapes. The embodiment depicted in FIG. 1A illustrates the shape of the relief member body 11 is comprised of a part circle, lesser in area than a semi-circle with the same diameter, adjacent a long side of a rectangle, wherein the diameter of the part circle is shorter than the long side of the rectangle and the meeting point of the circumference of the part circle with the long side of the rectangle is smooth and curved.

The relief member body 11 can be formed from a wide range of materials as discussed above. The embodiments depicted in the Figures are formed from an open cell foam. As is well illustrated in FIGS. 1A, 1B, 1C, 2, 6A, 7A, 7B, 7C, and 8A, in some preferred embodiments, the engagement means 12 is preferably partly formed or defined by virtue of a part-or full-thickness division 20 in relief member body 11. When the division 20 is full-thickness, it spans from the first relief surface 10A to the second relief surface 10B. Division 20 is preferably formed by a cut through the open cell foam.

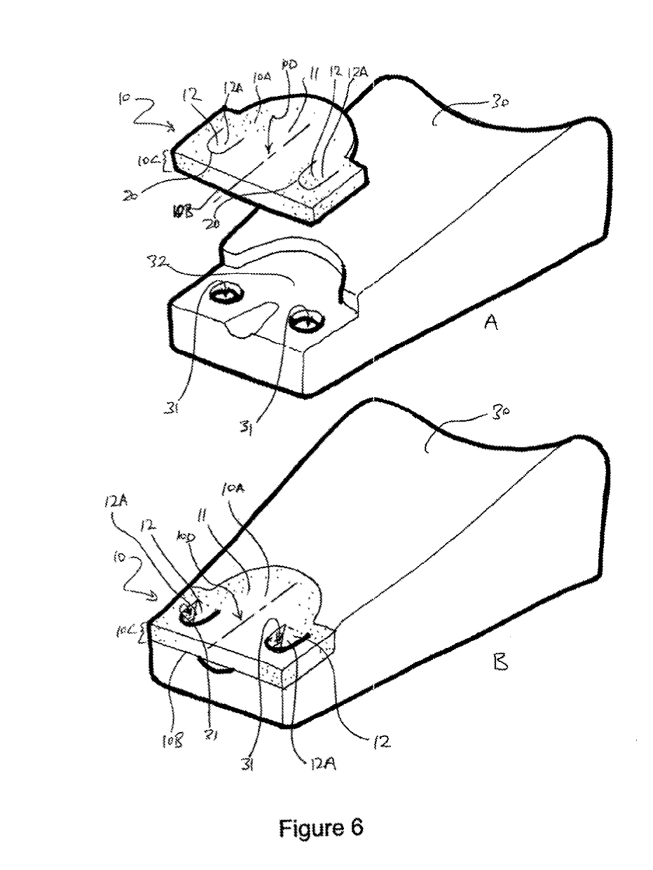

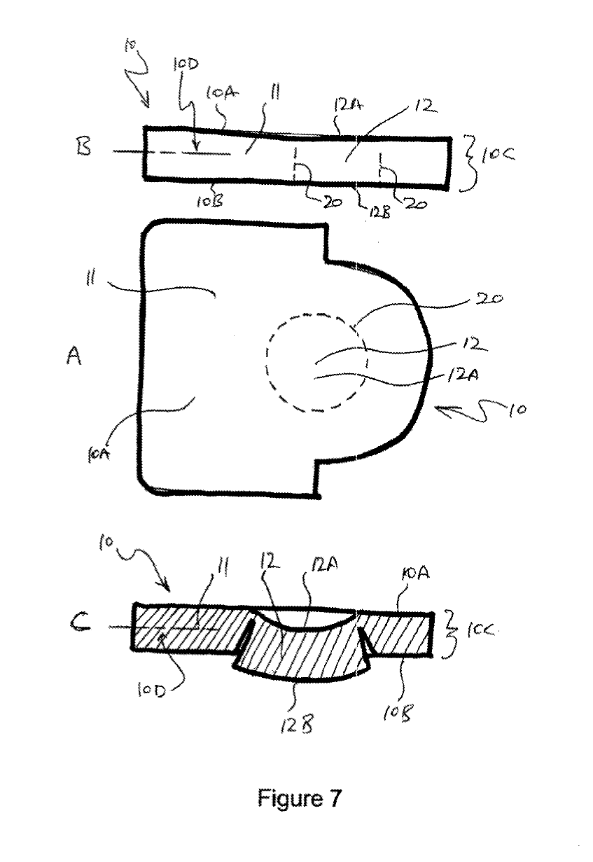

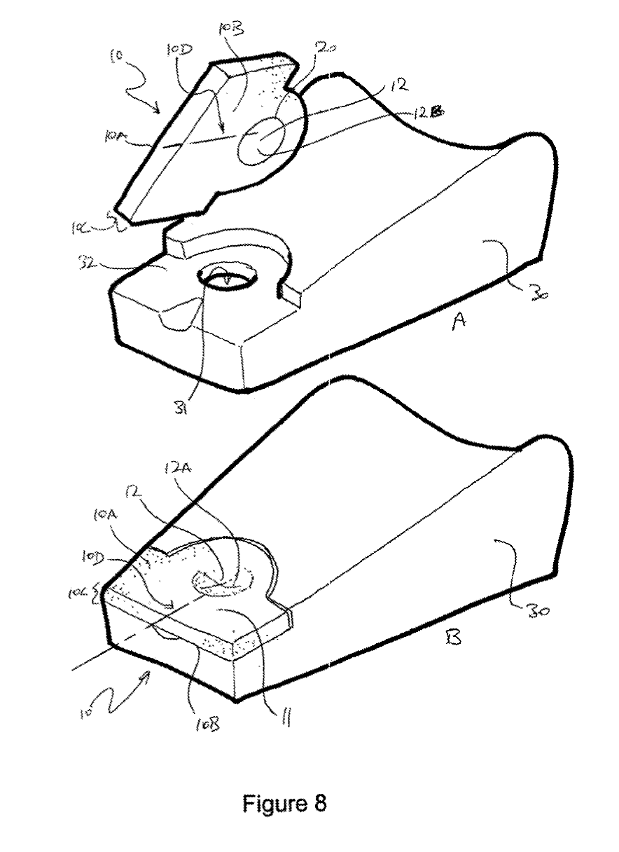

The shape of division 20 typically determines the shape, including the plan view shape, of engagement means 12 or of the first engagement surface 12A and/or of the second engagement surface 12B. As illustrated in FIG. 1A, the shape of division 20 is part circular and part triangular. The invention envisages a wide range of shapes for division 20. For example, the division illustrated in FIG. 2 is essentially part circular, the division illustrated in FIGS. 6A and 6B for each of engagement means 12 are part rectangular, part circular, and the division illustrated in FIGS. 7A, 8A and 8B is substantially circular.

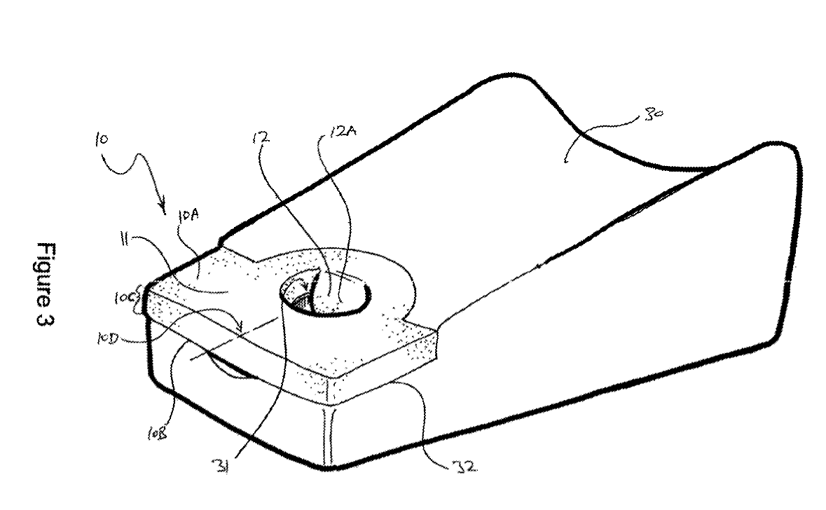

Turning to FIGS. 3, 4B, 5B, and 6B, it is noted that releasable engagement of the relief member 10 to orthosis 30 is preferably achieved when engagement means 12 is changed to the engagement configuration and advanced into an orifice, lacuna, or channel 31 in orthosis 30. The spatial arrangement of engagement means 12 in the engagement configuration releasably engages relief member 10 to orthosis 30 in the embodiments depicted in the Figures, by applying an outward pressure to an interior of the orifice, lacuna or channel 31. As is well illustrated in FIG. 4B, for example, releasable engagement of relief member 10 with orthosis 30 is preferably achieved taking advantage of the expansile properties of foam, which themselves may contribute to engagement means 12 being biased to the first configuration. This spatial arrangement of engagement means 12 in the engagement configuration applies an outward pressure to the interior of orifice, lacuna or channel 31 generating inertial forces capable of releasably retaining engagement means 12 in the orifice, lacuna or channel 31.

FIGS. 7A, 7B and 7C illustrate a relief member 10 according to another preferred embodiment of the invention. The shape of division 20 forming or defining engagement means 12 is substantially circular, and the division 20 is a partial-thickness division extending from relief surface 10B partially through body thickness 10C, stopping short of cutting through relief surface 10A. FIGS. 8A and 8B provide illustrations of a relief member 10 according to the embodiment depicted in FIGS. 7A, 7B and 7C respectively being positioned for engagement and then being engaged with orthosis 30.

As would be appreciated by persons skilled in the art, the size or ultimate volume of engagement means 12 relative to the size or volume of orifice, lacuna, or channel 31 may have a bearing on, or contribute to, the quality and quantity of engagement between relief member 10 and orthosis 30. Accordingly, as illustrated in FIGS. 8A and 8B, the greater the volume of the engagement means 12 in the engagement configuration, the greater the amount of engagement means 12 material that can be advanced (or forced) into orifice, lacuna or channel 31. In this way, a ‘force-fit’ scenario is created in which the engagement means 12 is effectively releasably wedged into the orifice, lacuna or channel 31.

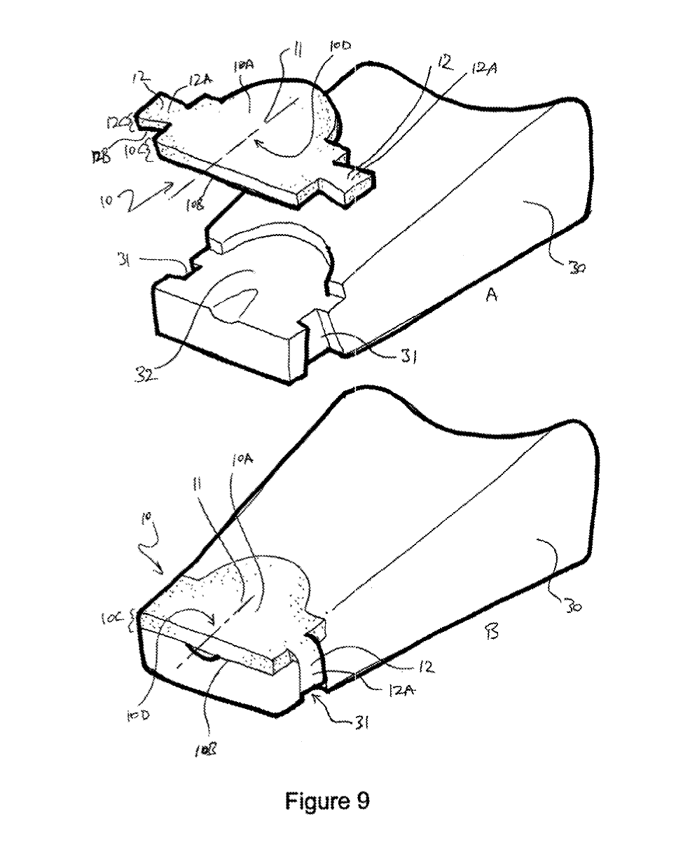

The relief member 10 depicted in FIGS. 9A and 9B is yet another embodiment according to the present invention. In this embodiment engagement means 12 are formed as an extension from the perimeter of the relief member body 11. When relief member 10 is placed in relief region 32 of orthosis 30, engagement means 12 are moved into the engagement configuration by being folded over the edge of orthosis 30 and then being advanced into channels 31. Engagement of relief member 10 according to this embodiment with orthosis 30 is illustrated in FIG. 9B.

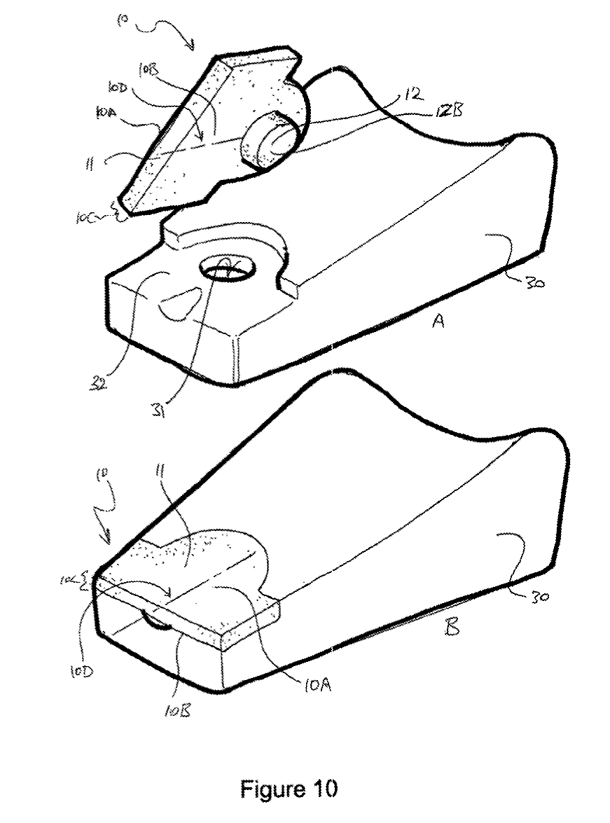

The relief member 10 depicted in FIGS. 10A and 10B is yet another embodiment according to the present invention. In this embodiment, engagement means 12 is formed as an extension from the second relief member surface 12B. Engagement means 12 is moved into the engagement configuration by compression. It is then advanced into orifice, lacuna or channel 31.

As indicated above, a relief member 10 according to the invention may have a plurality of engagement means 12. In such embodiments, the shapes of each of the engagement means 12 may be the same or different to one another.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A relief member for engaging a support member adapted to support a subject's body part, comprising:
a relief member body which includes a body thickness and comprises a relief member body plane disposed along a selected body axis; and
an engagement means integral with the relief member body and adapted to engage with a support member,
the engagement means being movable between a first configuration and an engagement configuration, the engagement means in the first configuration being disposed within extremity boundaries of the body thickness or is disposed substantially within the same plane as the relief member body plane, and the engagement means when in the engagement configuration extending outside the extremity boundaries of the body thickness or outside the relief member body plane to releasably engage the relief member to the support member; wherein the relief member body has a shape or configuration which substantially corresponds to a shape or configuration of at least a portion of a relief region formed in the support member to accommodate the relief member.

2. The relief member in accordance with claim 1 wherein the engagement means is an integral tongue or tab.

3. The relief member in accordance with claim 1 wherein the relief member body has a first relief surface and a second relief surface substantially opposite the first relief surface, and the body thickness is between the first and second relief surfaces and, in use, the first relief surface is adapted to rest on or adjacent the subject body part and the second relief surface is adapted to rest on or adjacent the support member.

4. The relief member in accordance with claim 1 wherein at least a portion of the relief member body is configured so as to contour at least a portion of the relief region when the relief member is located on or in the vicinity of the relief region.

5. The relief member in accordance with claim 1 wherein the relief member is key shaped and is adapted to contour at least a portion of the relief region which is correspondingly shaped.

6. The relief member in accordance with claim 1 wherein the relief member body is a composite shape made up of two or more shapes selected from a rectangle adjacent a semi-circle, a rectangle adjacent a part-circle, greater or lesser in area than a semi-circle with the same diameter.

7. The relief member in accordance with claim 1 wherein the relief member body and/or engagement means is formed from one or more materials selected from the group consisting of low density foam, high density foam, open cell foams, closed cell foams, rubber, solid gels, semi-solid gels, and/or a combination of two or more thereof.

8. The relief member in accordance with claim 1 wherein the relief member body is formed of one or more pliable enclosures, with each pliable enclosure adapted to receive, hold or expel relief member density content.

9. The relief member in accordance with claim 8 wherein the pliable enclosure is formed of one or more materials selected from the group consisting of polyurethane, resins, elastomers, polymers, copolymers, pliable plastics, leather, rubber, and a combination of two or more thereof.

10. The relief member in accordance with claim 8 wherein the relief member density content is selected from the group consisting of air, gas, gel, water, liquid, beads, foam particles or pieces, feathers, pulses or seeds of any kind, and a combination of two or more thereof.

11. The relief member in accordance with claim 1 wherein the relief member body is of variable density from one portion to another portion.

12. (canceled)

13. The relief member in accordance with claim 1 wherein the engagement means is partly formed by virtue of a part- or full-thickness division or cut through the relief member body.

14. The relief member in accordance with claim 13 wherein the division is full-thickness spanning a first to a second relief, or relief member body surface.

15. The relief member in accordance with claim 13 wherein the shape, or a part of the shape, of the division is selected from the group consisting of circular, ovular, rectangular, triangular, linear, elongated, elongated with a portion which is part circular, part ovular, part rectangular, part triangular, part linear, a composite shape comprising two or more shapes, and combinations of thereof.

16. The relief member in accordance with claim 1 wherein releasable engagement of the relief member to the support member is achieved when the engagement means is changed to the engagement configuration and advanced into an orifice, lacuna, or channel in the support member.

17. The relief member in accordance with claim 15 wherein the releasable engagement is achieved as the engagement means is caused to advance into the orifice, lacuna, or channel of the support member to take up the engagement configuration and expansile properties of the foam generate a spatial arrangement for the engagement means which applies outward pressure to at least a portion of the orifice, lacuna, or channel interior.

18. The relief member in accordance with claim 1 wherein the engagement means is formed substantially centrally on the relief member body or the engagement means is formed toward the perimeter of the relief member body.

19. (canceled)

20. The relief member in accordance with claim 1 wherein the engagement means is generally biased toward the first configuration.

21. (canceled)

22. (canceled)

23. A method of forming a relief member according to claim 1, the method comprising the steps of:
forming a relief member body; and
forming a division in the relief member body adapted to form at least part of an engagement means.

24. (canceled)

25. A method of releasably engaging a relief member to an object, the method comprising the steps of:
providing a relief member in accordance with claim 1;
providing an orifice, lacuna, or channel in the object; and
advancing the engagement means into the orifice, lacuna, or channel so as to adopt the engagement configuration thereby releasably engaging the relief member to the object.

26. (canceled)
